(19) United States
(12) Patent Application Publication
(10) Pub. No.: US 2010/0152239 A1
(43) Pub. Date: Jun. 17, 2010

(54) Method to Improve Survival in Sepsis

(76) Inventors: Luis Ulloa, Jackson Heights, NY (US); Fei Chen, Denville, NJ (US); Bolin Cai, Newark, NJ (US)

Correspondence Address: UMDNJ;Office of Patents and Licensing, Suite 2100, One Worlds Fair Drive, Somerset, NJ, 08873, US

(21) Appl. No.: 12/617,236

(22) Filed: Nov. 12, 2009

Related U.S. Application Data
(60) Provisional application No. 61/199,118, filed on Nov. 12, 2008.

(51) Int. Cl. A61K 31/4545; A61P 29/00

(52) U.S. Cl. 514/318

(57) ABSTRACT

This invention relates to the use of an α7nAChR-agonist such as GTS to control systemic inflammation during resuscitation following severe trauma such blood loss because of severe hemorrhage.

This application claims priority to U.S. provisional application No. 61/199,118, filed Nov. 12, 2008, the disclosure of which is hereby incorporated by reference in its entirety.

The invention was made with U.S. government support, and the U.S. Government may have certain rights in the invention, as provided for by the terms of Grant ID Number GM084125 awarded by the National Institute of Health and Grant ID USARMC#05308004 awarded by the US Army.

The innate immune system is an essential component of our defenses to trauma and injury. But at the same time, it is one of the principal causes of morbidity and mortality in critical care. This outcome is especially dramatic in severe hemorrhage, because after losing over 50% of the blood volume, the system is unable to re-establish tissue perfusion. Thus, resuscitation fluids are classically designed to restore circulatory volume and tissue perfusion but they fail to prevent inflammatory responses during resuscitation. Characteristic inflammatory cytokines such as tumor necrosis factor (TNF) and high mobility group B protein-1 (HMGB1) contribute to lethal cardiovascular shock during resuscitation. TNF diffuses in the bloodstream and produces a fatal cardiovascular collapse. TNF is a sufficient and necessary mediator of ‘hemorrhagic shock’ because: (i) it is found in patients and experimental models of ‘hemorrhagic shock’; (ii) TNF may contribute to the lethality of hemorrhagic shock; and (iii) TNF neutralization attenuates cardiovascular shock. In addition to TNF, recent studies indicated that HMGB1 plays an important role in hemorrhagic shock. HMGB1 was originally identified as a nuclear DNA-binding protein that functions as a structural co-factor. However, during cellular damage or necrosis, HMGB1 is liberated into the extracellular milieu where it functions as an inflammatory cytokine. Extracellular HMGB1 sustains inflammatory responses producing abrupt cardiac standstill, intestinal derangement and acute lung injury. For these reasons, there is a major interest in inhibiting the production of these actors during resuscitation.

It has now been found that GTS is a promising supplement to control systemic inflammation during resuscitation and improve survival in severe hemorrhage.

Further aspects of this invention are more fully described below in conjunction with the accompanying Figures wherein:

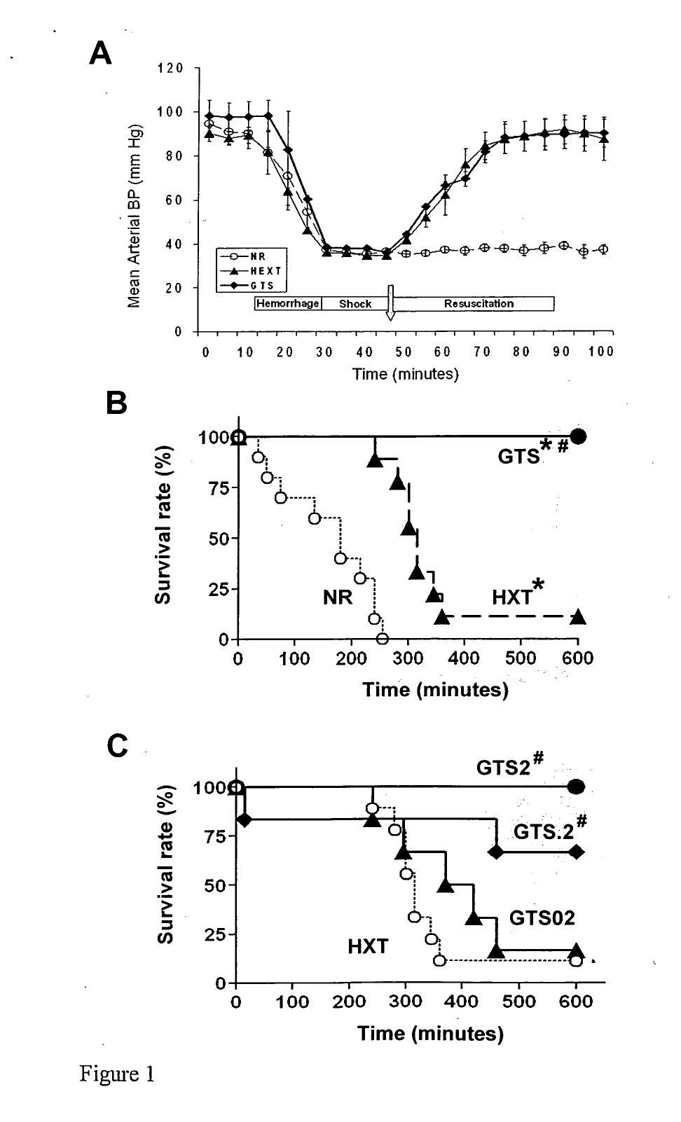

FIG. 1 shows resuscitation with alpha7nAChR-agnoist improves survival in severe hemorrhage;

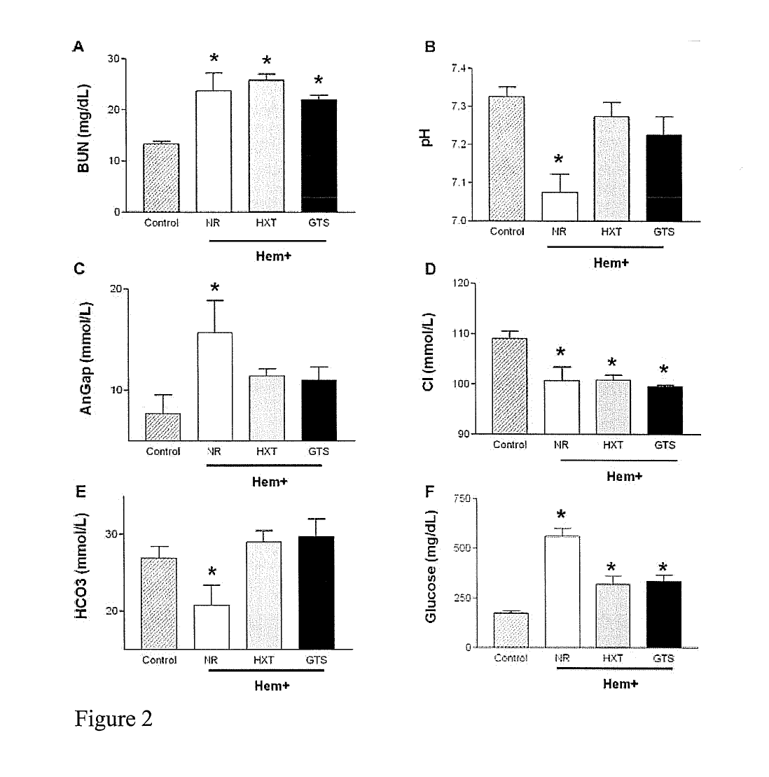

FIG. 2 shows blood chemistry analyzes during resuscitation;

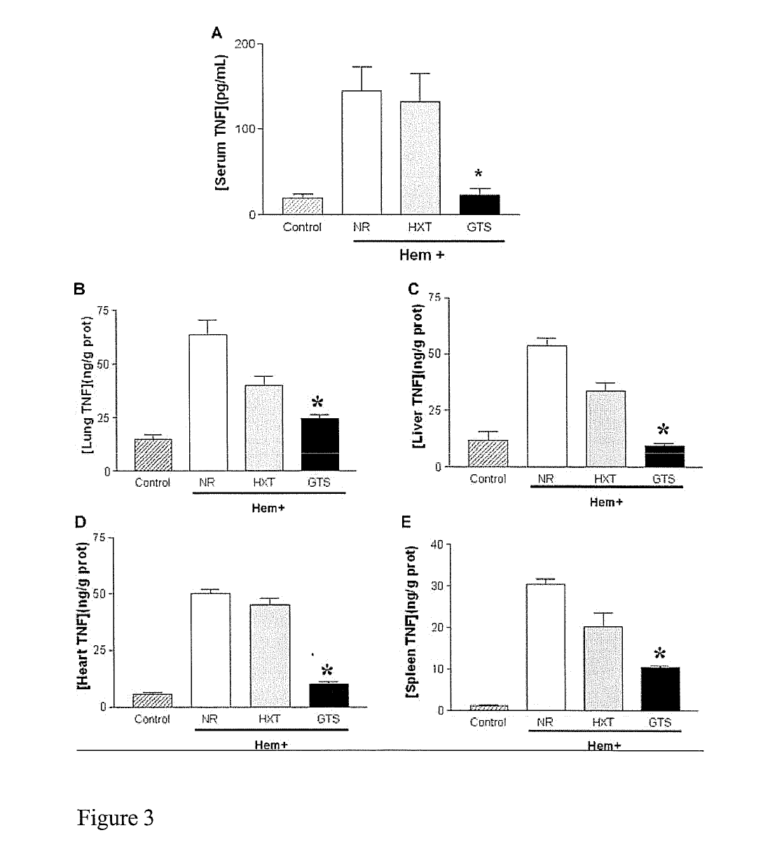

FIG. 3 shows GTS inhibits systemic TNF response during resuscitation;

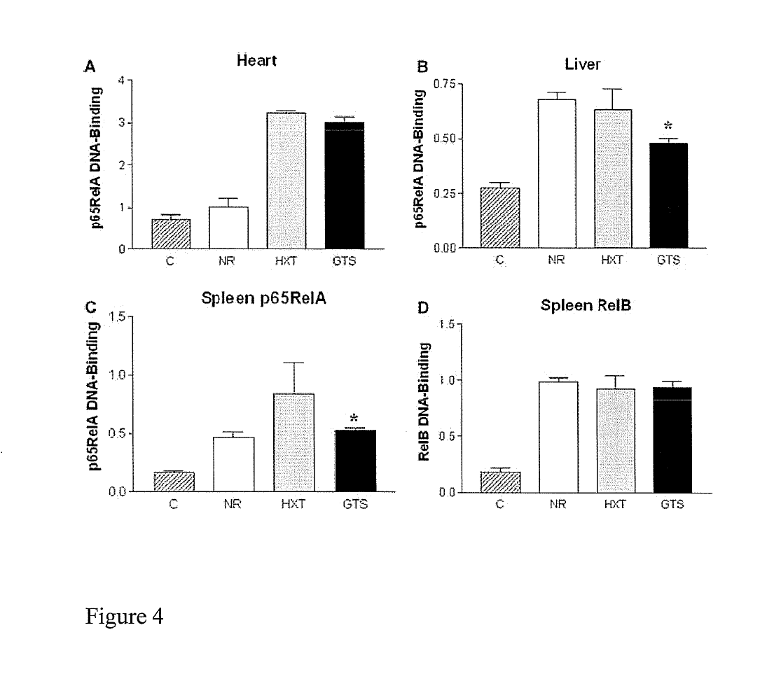

FIG. 4 shows GTS inhibits splenic RelA but not RelB NF-κB in vivo during resuscitation;

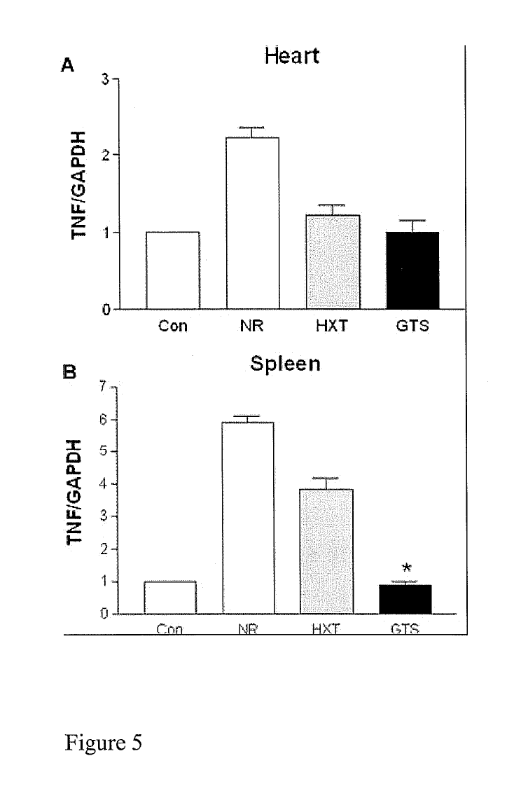

FIG. 5 shows GTS inhibits TNF transcription in the spleen during resuscitation;

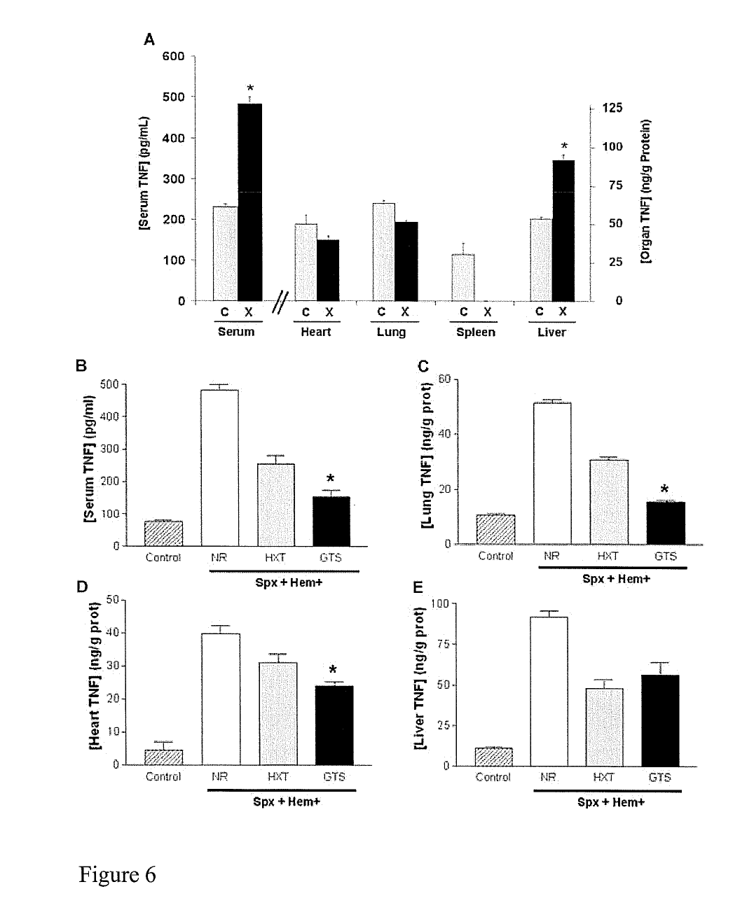

FIG. 6 shows GTS attenuates systemic TNF levels in splenectomized animals after resuscitation; and

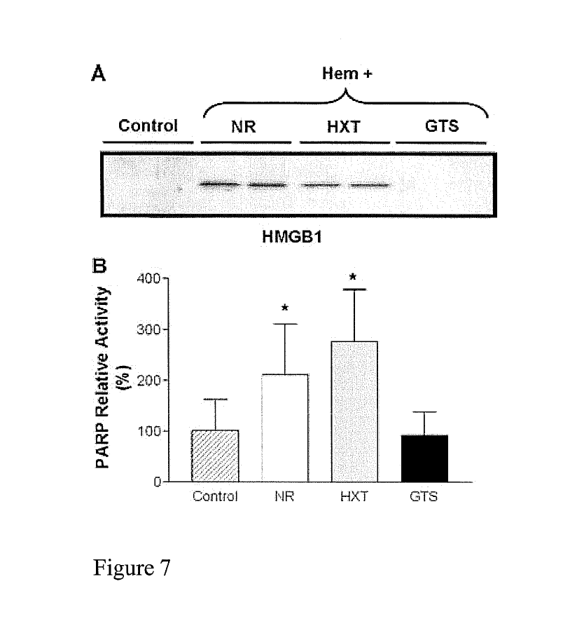

FIG. 7 shows alpha7nAChR-agonist inhibits systemic HMGB1 response.

The nervous system is an important regulator of the immune system, and neuronal anti-inflammatory mechanisms have been selected by evolution to modulate inflammatory responses. These mechanisms can provide a major advantage for novel pharmacological anti-inflammatory strategies to control systemic inflammation. We have reported that the parasympathetic nervous system restrains systemic inflammation via the vagus nerve. Electrical stimulation of the vagus nerve inhibits serum TNF levels in wild-type but not in alpha7nAChR-knockout mice. However, the anti-inflammatory potential of this mechanism is limited by the capacity of the vagus nerve to release acetylcholine and the neuronal innervation of specific organs. Thus, we studied the anti-inflammatory potential of acetylcholine, the principal neurotransmitter of the vagus nerve. Acetylcholine inhibits the production of both TNF and HMGB1 in macrophages via the alpha7 nicotinic acetylcholine receptor (nAChR). In vivo, treatment with nicotine, a more selective cholinergic agonist, inhibits serum TNF and HMGB1 levels and improves survival in experimental models of systemic inflammation. Nicotine has been used in several clinical trials including inflammatory bowel disorders, but its therapeutic potential is limited by its collateral toxicity. We proposed that alpha7nAChR-agonists could avoid the collateral toxicity of nicotine. GTS is a characteristic alpha7nAChR-agonist, has been used in clinical trials and has proven to be less toxic than nicotine. GTS has been used in clinical trials to target neuronal alpha7nAChR in the brain of patients with Alzheimer's disease. These studies showed a limited effect of GTS on the central nervous system potentially due to its limited ability to cross the blood-brain barrier. From an immunological perspective, this characteristic is an advantage to use GTS in the periphery and avoid secondary effects on the central nervous system. In contrast to nicotine, GTS has no effect on locomotor activity or on dopamine turnover indicating that it is less toxic than nicotine. Thus, we reasoned that GTS would represent a promising supplement to control systemic inflammation during resuscitation.

Recently, we reported that similar to vagus nerve stimulation, nicotine inhibits TNF and HMGB1 production from macrophages via alpha7nAChR. We have now tested the ability of the alpha7-agonist, GTS, to control systemic inflammation during resuscitation. Hextend was used as the control resuscitation fluid because recent studies indicate that it prevents multiple organ injury and improves short-time survival as compared with saline solution. Hextend is a novel plasma volume expander containing 6% hydroxyethyl starch in Ringer's lactate.

Hetastarch creates oncotic pressure, which is normally provided by blood proteins and permits retention of intravascular fluid. Our current study indicates that resuscitation with Hextend supplemented with GTS rescued all the animals from lethal hemorrhage and improved survival in a concentration-dependent manner. GTS inhibited the production of cardiodepressant factors including both HMGB1 and TNF. It was particularly effective at inhibiting TNF production in the spleen and modulating splenic NF-kB during resuscitation. Unlike non-specific nicotinic agonists, GTS inhibited systemic TNF levels in both control and splenectomized animals. Resuscitation with GTS inhibited hepatic poly(ADP-ribose) polymerase and systemic HMGB1 levels.

Conventional resuscitation fluids are designed to re-establish blood pressure and tissue perfusion, but they fail to prevent lethal inflammatory responses during resuscitation. For instance, resuscitation with Hextend re-established blood pressure and tissue perfusion, but still 90% of the animals died within the first 6 hrs after hemorrhage. Here, we report that all the animals resuscitated with Hextend supplemented with GTS survived lethal hemorrhagic shock. Our results are particularly significant for three reasons. First, animals were subjected to severe hemorrhage with approximately 50% of blood loss where all the control animals died within the first 5 hrs after hemorrhage. Second, shed blood was considered lost and it was not reinfused. Our animals were treated with a small volume of resuscitation of 15 ml/kg that represents approximately 50% of the shed blood volume. Moreover, our studies use Hextend as control solution. This is a critical consideration as recent studies indicate that resuscitation with Hextend prevents multiple organ injury and improves survival as compared with saline. Third, the animals were followed for up to 7 days to analyze total survival including late deaths. GTS did not affect basic physiological parameters. Both resuscitation with Hextend or Hextend with GTS re-established blood pressure, prevented metabolic acidosis and hyperglycaemia. The most significant result correlating with the therapeutic potential of GTS was its anti-inflammatory potential to restrain systemic TNF and HMGB1 levels.

Physiological anti-inflammatory mechanisms represent efficient systems selected by evolution to control inflammation and provide significant opportunities for novel anti-inflammatory strategies. The vagus nerve restrains systemic inflammation in wild-type but not in alpha7-knockout mice. Both, acetylcholine and nicotine control the production of inflammatory cytokines in macrophages via the alpha7 nicotinic receptor. Treatment with nicotine inhibits serum TNF and HMGB1 levels and improves survival in experimental models of systemic inflammation. Despite its clinical implications, the anti-inflammatory potential of cholinergic agonists is underestimated due to the collateral toxicity of nicotine including addiction, tachycardia and arrhythmia. We proposed that alpha7nAChRagonists might represent a promising pharmacological strategy to control systemic inflammation and avoid collateral toxicity. This hypothesis is consistent with recent studies indicating that individual cholinergic receptors mediate specific properties of nicotine. Thus, α7nAChR-agonists are expected to avoid nicotine induced addiction, locomotor activity, tumourigenesis], autonomic dysfunction or allodynia, which appear to be mediated by other nicotinic receptors namely β2nAChR, α3nAChR, α4nAChR or α5nAChR, respectively. GTS (3-[(2,4-dimethoxy)benzylidene]-anabaseine) is a characteristic alpha7nAChR-agonist, already used in clinical trials and proven less toxic than nicotine. GTS had no effect on locomotor activity in mice or dopamine turnover in rats. In contrast, nicotine produced a biphasic effect on locomotor activity. These results prompt the potential use of GTS to avoid collateral toxicity of unspecific agonists.

GTS inhibited systemic HMGB1 levels during resuscitation. Originally described as an intracellular protein, HMGB1 can be released into the extracellular milieu where it functions as a proinflammatory cytokine. Extracellular HMGB1 acts as a pro-inflammatory cytokine that activates immune cells and sustains inflammatory responses contributing to abrupt cardiac standstill, acute lung injury and intestinal derangement. HMGB1 appears to be a pharmacologic target for hemorrhagic shock as: (a) Serum HMGB1 levels are increased in patients with hemorrhagic shock; and (b) Inhibition of HMGB1 activity with neutralizing antibody significantly decreased liver damage after ischemia and reperfusion. Our previous studies supported HMGB1 as a late pharmacologic target for systemic inflammation because it appears in the serum at 18-24 hrs after the induction of sepsis. However, this study now shows ‘early’ serum HMGB1 levels at 2 hrs after hemorrhage. Recent studies also reported ‘early’ extracellular HMGB1 release after hepatic ischemia/reperfusion. These differences between ‘late’ secretion in sepsis and ‘early’ release in hemorrhage may be explained by two different mechanisms of HMGB1 release/production. The first mechanism may be a time-consuming ‘active secretion’ from immune cells to act as a pro-inflammatory cytokine during an immunologic challenge such as sepsis. The second mechanism may be the ‘passive release’ of HMGB1 from damaged or necrotic cells during hemorrhage. In this hemorrhagic scenario, HMGB1, an intracellular protein, represents an optimal signal to recognize tissue damage and initiate reparative responses. From an immunological perspective, HMGB1 represents a characteristic ‘necrotic marker’ or damage associated molecular pattern (DAMP) molecule. This emerging family of specific intracellular proteins represents optimal chemotactic markers selected by the innate immune system to recognize tissue damage and initiate reparative responses. From a pathological perspective, HMGB1 can be used as a marker for cellular necrosis and tissue injury. In this sense, resuscitation with Hextend with GTS, but not Hextend alone, prevented serum HMGB1 levels supporting its therapeutic effects.

Our previous studies indicate that nicotine inhibits endotoxininduced p65NF-κB activation in RAW264.7 macrophage cells via alpha7nAchR. Here, we report that in vivo, GTS, inhibits p65NF-κB activation in the spleen but not in the heart during resuscitation as compared with Hextend alone. However, GTS is no different than NR in altering p65RelA, and both are less than Hextend, yet the pattern of TNF and p65RelA did not correlate as would be expected. A potential explanation is that the expression of alpha7nAChR may limit the effect of GTS to particular cells. This effect could be overlooked by analyzing total extract of organs. Future studies are needed to study the regulation of p65RelA by GTS in specific cell types. This mechanism appears to be specific for p65RelA NF-κB as RelB was not affected by GTS. Hemorrhage induced a significant activation of both pathways but conventional resuscitation further activated p65RelA but not RelB. Because GTS specifically inhibited p65RelA, it can be used to further study the different implications of the conventional p65RelA and the alternative RelB NF-κB pathway in resuscitation. The activation of p65RelA NF-κB in the heart and the spleen during resuscitation did not correlate with increased mRNA TNF levels in these organs. These potential discrepancies can be due, at least in part, to the implications of other factors required for TNF transcription. In addition to NF-κB, GTS also inhibited the activation of poly(ADP-ribose) polymerase (PARP) providing the first evidence of the regulation of PARP by the alpha7nAChR. This effect has significant implications because PARP contributes to hemorrhagic shock as PARP inhibition provides marked survival benefit and protects from organ injury. Of special note is the correlation between PARP inhibition and systemic HMGB1. PARP inhibition can explain the regulation of systemic HMGB1 as PARP contributes to cell death via necrosis and PARP inhibitors prevent HMGB1 release in necrotic cells. Furthermore, because PARP also regulates NF-κB activation and inflammatory cytokines, PARP inhibition may mediate the anti-inflammatory effects of cholinergic agonists via the alpha7nAChR.

Our previous studies indicated that the spleen is a major source of serum TNF and inhibition of TNF production in the spleen can prevent cardiovascular shock in endotoxemia. The vagus nerve and non-specific cholinergic agonists prevent systemic inflammation during endotoxemia in normal but not in splenectomized animals. These results have significant clinical implications suggesting that non-specific cholinergic agonists may not be efficient in patients with injured or compromised spleen. In sepsis, splenectomy moderated systemic TNF levels and abolished the anti-inflammatory potential of unspecific cholinergic agonists. In contrast to septic shock, splenectomy failed to prevent systemic inflammation in hemorrhagic shock. Actually, serum TNF levels during hemorrhagic shock were significantly higher in splenectomized than in normal animals. These higher serum TNF levels in splenectomized animals correlated with higher TNF levels in the liver. One potential explanation is that splenectomy may prevent the regulation of hepatic TNF production induced by the splenic release of anti-inflammatory cytokines such as TGF-β into the splenic and portal vein. In any case, these results reveal a fundamental difference between the systemic inflammatory response in experimental sepsis and hemorrhage. Indeed, Hextend reduced splenic TNF in normal animals (FIG. 3) and yet there was little influence upon serum and cardiac TNF levels. These results may argue against a splenic TNF etiology as the mechanism for mortality, unless there is a TNF threshold, below which one may observe survival benefit. Resuscitation with Hextend attenuates systemic TNF levels in splenectomized but not in control rodents. Similar to nicotine, GTS induced a stronger anti-inflammatory effect in normal rodents. But unlike nicotine, GTS efficiently attenuated serum TNF levels and prevented systemic inflammation in both normal and splenectomized animals. From a pharmacological perspective, these results suggest that GTS could provide a significant advantage as compared with unspecific cholinergic agonists to control systemic inflammation including patients with injured or compromised spleen. Taken together, our studies suggest that GTS could restrain the production of critical inflammatory and cardiodepressant factors during resuscitation.

### Materials and Methods

### Animal Experiments

Adult male Sprague-Dawley (350-450 g) rats were purchased from Harlen Sprague-Dawley (Indianapolis, Ind., USA) and allowed to acclimate for 7 days, housed at 25° C. on a 12-hr light/dark cycle. Animals were randomly grouped and assigned to a specific experiment. Investigators were blinded to the experimental treatment.

### Hemorrhage

Animals were anaesthetized by inhalation of isoflurane (5% induction, 2% maintenance; Minrad, Buffalo, N.Y., USA) and subjected to surgical catheter placement into the femoral artery and vein under sterile conditions. To avoid blood clot and maintain catheter patency, the catheters were flushed with 1% heparin solution immediately before placement. Heparin was not used in vivo during the experiment or observation period in agreement with previous studies. After the catheter implantation, the blood pressure and the heart rate were recorded for 15 min. to establish a physiological baseline prior the hemorrhage procedure. Then, the animals were subjected to hemorrhage over 15 min. to reach a mean arterial blood pressure (MAP) of 35-40 mmHg and subsequent maintenance of this blood pressure by continued blood withdrawal for another 15 min. After the shock phase, resuscitated animals received specific resuscitation treatment over 40 min. with a total volume of 15 ml/kg (equivalent to 1000 ml of Hextend in a 70 kg man). The femoral artery was catheterized with a catheter R-FAC (Braintree Scientific, Inc., Braintree, Mass., USA) previously flushed with 1% heparin in 0.9% saline to allow spontaneous bleeding due to arterial blood pressure. Shed blood was considered lost and it was not reinfused. After resuscitation treatment, the animals were kept anesthetized for 2 hrs to record heart rate, blood pressure and blood chemistry. The control animals were also anesthetized with isoflurane for blood collection. Then, the anesthesia was stopped and the animals were allowed to recover from anesthesia and housed individually in regular cages. Splenectomy was performed as we described previously. Briefly, the spleen was identified following a midline laparotomy incision, and removed after appropriate blood vessel ligation. Sham animals underwent laparotomy without splenectomy.

### Blood Chemistry and Cytokine Measurement

Activation Blood was collected at 2 hrs after the hemorrhagic shock and analyzed using the i-Stat blood analyzer (Abbot Laboratories, IL, USA). Lung function was assessed by analyzing blood gases including total and partial carbon dioxide (TCO₂, PCO₂), bicarbonate (HCO₃), pH and the base excess of extracellular fluid (BEecf). The organ function tests include anion sodium, potassium, chloride, the anion gap (AnGap), total plasma protein (TP), and blood urea nitrogen (BUN). Blood chemistry also included glucose, hematocrit and hemoglobin. The systemic inflammatory status was assessed by analyzing critical pro-inflammatory cytokines and cardiodepressant factors such as TNF and HMGB1. The major organs were collected and immediately sliced and washed with PBS at 4° C. TNF concentrations in serum and major organs were determined by ELISA (R&D Systems, Inc., Minneapolis, Minn., USA). HMGB1 was analyzed by Western blot as previously described.

### NF-κB and PARP Analyses

Homogenates were normalized by protein concentration and the activation of IκBα was analyzed by Western blot by using anti-IκBα polyclonal antibody ab7217 and anti-phosphorylated-IκBα(phospho S32+S36) monoclonal antibody [clone39A1431] ab12135 (Abcam; Cambridge, Mass., USA). Transcriptional activity of splenic p65RelA was analyzed by TransAM DNABinding ELISA (Active Motif; Cambridge, Mass., USA) following manufacturer's instructions. The results were confirmed by EMSA from nuclear extracts performed as we previously described. PARP activity in organ homogenates was analyzed using a commercially available kit (R&D Systems, Cat#4677-096K) following the manufacturer's instructions. Protein levels were measured using the Bradford method (BIO-RAD) and PARP activity was normalized to protein content.

### Statistical Analyzes

All data in the figures and text are expressed as mean±standard deviation (SD). Statistical analyzes were performed using the one-way ANOVA with multiple pairwise comparisons with the Bonferroni's adjustment for multiple hypothesis testing. The Student's t-test was used to compare mean values between two experimental groups. Statistical analyzes of survival were determined using the logrank test. P-values<0.05 were considered statistically significant.

### Results

Alpha7nAChR-Agonist Improved Survival During Resuscitation

See FIG. 1 which shows resuscitation with alpha7nAChR-agonist improves survival in severe hemorrhage. Adult male Sprague-Dawley hemorrhagic rats received no resuscitation (NR) or were resuscitated with 15 ml/kg (i.v.) Hextend (HEXT) or Hextend supplemented with GTS (GTS). (A) Arterial blood pressure was recorded during hemorrhage and the resuscitation treatment. (B) All animals without resuscitation treatment (NR; n=10) died within the first 5 hrs after hemorrhage with an average time of death of 161±26 min. Resuscitation with Hextend allowed the animals to restore normal blood pressure and tissue perfusion, and yet 90% of the animals died (n=9). Resuscitation with Hextend supplemented with GTS (GTS) statistically protected all the animals (n=7/group). (C) Treatment with 2 (GTS2), 0.2 mg/kg (GTS.2), but not 0.02 mg/kg (GTS02) GTS induced a statistically significant protection as compared with Hextend solution alone. P<0.05 survival logrank test versus NR (*) or HXT(#).

Hemorrhagic shock required withdrawing 21±4.3 ml blood/kg body weight, and the maintenance of that blood pressure for another 15 min. required another 7±2.7 ml blood/kg body weight. Animals without resuscitation treatment (NR; n=10) did not re-establish normal blood pressure (FIG. 1A), and all animals died within the first 5 hrs after hemorrhage with an average time of death of 161±26 min. (FIG. 1B). Resuscitation with 15 ml/kg Hextend allowed the animals to recover normal blood pressure and re-establish tissue perfusion, but still, 90% of the animals died within the first 5 hrs after hemorrhagic shock (P<0.001, logrank test Hextend versus NR; n=9). Resuscitation with Hextend supplemented with 350 uM GTS rescued all the animals from lethal hemorrhage (P<0.05, logrank survival test versus Hextend; n=7). GTS improved survival in hemorrhagic shock in a concentration-dependent manner (FIG. 1C). Treatment with 2 or 0.2 mg/kg but not 0.02 mg/kg GTS induced a statistically significant protection as compared with Hextend solution alone. Treatment with 0.2 mg/kg GTS protected 67% of the animals with hemorrhagic shock, whereas 0.02 mg/kg GTS protected 17% of the animals (n=7/group). Several animals died during the hemorrhagic shock and three animals died during the resuscitation treatment. One animal died during resuscitation with Hextend alone, one during resuscitation with Hextend+GTS at 2 mg/kg and another one during resuscitation with Hextend+GTS 0.2 mg/kg. All these animals were excluded of our statistic analyzes as many of these deaths were attributed to a particular problem with the anesthesia system. Our analyses excluded the animals that died during the surgical procedure and counted only those animals that completed the resuscitation treatment. All nonsurvival animals died within the first 8 hrs after hemorrhagic shock. All the animals that passed this critical period survived the hemorrhagic shock, and no late deaths were found, although the animals were followed for up to 7 days. These results suggest that GTS induced a lasting protection and it did not merely delay the pathologic onset.

Alpha7nAChR-Agonist Prevented Systemic Inflammation During Hemorrhage

Characteristic pathological markers of hemorrhage were assessed by blood chemistry. Animals without resuscitation were characterized by uremia, metabolic acidosis and hyperglycemia (FIG. 2, Blood chemistry analyses during resuscitation. Blood from control adult male Sprague-Dawley rats, or hemorrhagic animals without resuscitation treatment (NR) or resuscitated with 15 ml/kg (i.v.) Hextend (HXT) or Hextend containing GTS (GTS) was collected at 2 hrs after the hemorrhagic load to analyze (A) blood urea nitrogen (BUN), (B) pH, (C) the anion gap (AnGap), (D) chloride (Cl), (E) bicarbonate (HCO₃) and (F) Glucose. * represents P<0.01 versus control (n=4/group; one-way ANOVA with Bonferroni's corrections)). Animals without resuscitation showed characteristic high levels of BUN. Although resuscitation with Hextend or Hextend supplemented with GTS improved survival, they did not prevent uremia (FIG. 2A). Hemorrhagic animals without resuscitation also showed metabolic acidosis that was associated with elevated anion gap (AnGap) levels, low levels of chlorine and bicarbonate (FIG. 2B-E). Both resuscitation with Hextend or Hextend supplemented with GTS prevented metabolic acidosis and re-established normal values of AnGap and bicarbonate but not hypochloremia. Hemorrhage also causes a characteristic hyperglycemic response that was significantly prevented by both resuscitation with Hextend and Hextend supplemented with GTS (FIG. 2F). Together, these results indicated that the therapeutic potential of GTS was not due to these mechanisms.

Because our previous studies indicated that the vagus nerve controlled systemic TNF responses via alpha7nAChR, we reasoned that the alpha7-agonist will produce a similar effect. Indeed, the most significant effects of GTS were in inhibiting systemic TNF levels. (FIG. 3 GTS inhibits systemic TNF response during resuscitation. Blood and organs from control or haemorrhagic animals without (NR) or with resuscitation with Hextend (HXT) or Hextend supplemented with GTS (GTS) were analysed at 2 hrs to determine TNF protein concentration in the (A) serum, (B) lung, (C) liver, (D) heart and (E) spleen. * represents P<0.05 versus HXT (n=4/group; one-way ANOVA with Bonferroni's corrections). Hemorrhage induced a systemic TNF response that was not affected by resuscitation with Hextend (FIG. 3A). However, resuscitation with Hextend containing GTS inhibited this lethal response, and these animals had circulating TNF levels similar to those in control mice. Albeit these results appear not to be statistically significant with Bonferroni's corrections, resuscitation with Hextend alone reduced TNF levels in the lung, liver and spleen, though not to the extent of GTS, and yet there was little influence upon serum and cardiac TNF. Because the effects of Hextend have been previously described, we have performed conservative statistical analyzes to focus on the effects of Hextend+GTS as compared with Hextend alone. As compared with Hextend, resuscitation with GTS blunted TNF levels in all of the organs, but particularly in the spleen, liver and the heart. Because our studies indicated that both acetylcholine and nicotine inhibit NF-κB in macrophages via alpha7nAChR, we wonder whether GTS would inhibit this pathway during resuscitation. Canonical p65RelA and alternative RelB binding to DNA were analyzed by TransAM DNA-Binding ELISA (FIG. 4, GTS inhibits splenic RelA but not RelB NF-κB in vivo during resuscitation. Organs from control (C) or hemorrhagic animals without (NR) or with resuscitation with Hextend (HXT) or Hextend supplemented with GTS (GTS) were collected at 2 hrs to analyze NF-κB during resuscitation in the (A) heart, (B) liver and (C, D) spleen. Both canonical p65RelA and (A-C) alternative RelB (D) NF-κB binding to DNA were analyzed by TransAM DNA-Binding ELISA. Results are provided in arbitrary but relative units among the different samples and organs. * represents P<0.05 versus HXT=5/group; one-way ANOVA with Bonferroni's corrections)). Nuclear extracts from major organs were collected at 2 hrs after hemorrhage, which is approximately 30 min. before the average time of death for the control animals without resuscitation treatment. Resuscitation with Hextend did not affect p65RelA in the liver but increased its activation in the heart and the spleen. Hemorrhage induced a significant activation of both pathways but conventional resuscitation further activated p65RelA but not RelB. These results concur with recent studies indicating that resuscitation promotes systemic inflammatory responses. Resuscitation with Hextend supplemented with GTS inhibited p65RelA NF-κB in most of the organs but not in the heart. Again, the most significant results with GTS were found in the spleen. These effects were specific for p65RelA NF-κB as RelB was not affected by either resuscitation with Hextend or GTS (FIG. 4D).

Our results suggest that GTS may inhibit systemic and cardiac TNF levels by inhibiting TNF transcription in the spleen similar to what we described in experimental models of sepsis. We analyzed mRNA TNF levels in the heart and the spleen by real-time PCR (FIG. 5, GTS inhibits TNF transcription in the spleen during resuscitation. Organs from control or hemorrhagic animals without or with resuscitation were analyzed at 2 hrs after hemorrhage. TNF transcription in the (A) heart and (B) spleen was analyzed by real-time PCR. * represents P<0.05 versus HXT (n=4/group; one-way ANOVA with Bonferroni's corrections)). Hemorrhage increased mRNA TNF levels in both the heart and the spleen as analyzed by real-time PCR. As compared with Hextend, GTS inhibited NF-κB and decreased mRNA TNF levels in the spleen but not in the heart. In order to determine whether the inhibition of splenic TNF is sufficient to account for the anti-inflammatory potential of GTS, we analyzed the pharmacological implications of the spleen during resuscitation.

### Pharmacological Implications of the Spleen in Resuscitation

Our previous studies indicated that both vagus nerve stimulation and treatment with nicotine attenuated systemic TNF levels in sham but not in splenectomized animals. We studied the pharmacological implications of the spleen during resuscitation using splenectomized animals with hemorrhage. (FIG. 6, GTS attenuates systemic TNF levels in splenectomized animals after resuscitation. (A) Serum TNF levels were analyzed in blood collected from non-splenectomized (c) or splenectomized (x), hemorrhagic animals. Higher serum TNF levels in splenectomized animals correlated with higher TNF levels in the liver but not in the lung or the heart. * represents P<0.05 (Student's t-test). Hemorrhagic splenectomized animals were subjected to the different treatments and TNF concentrations were determined in the (B) serum, (C) lung, (D) heart and (E) liver. * represents P<0.05 versus HXT (n=4/group; one-way ANOVA with Bonferroni's corrections)). Blood chemistry analyzes showed that hemorrhage in splenectomized animals induced similar uremia, metabolic acidosis and hyperglycemia as compared to normal animals (data not shown). Again, the most significant effects of GTS were found in systemic TNF levels. Unlike systemic inflammation during endotoxemia, splenectomy actually exacerbated serum TNF levels during resuscitation (FIG. 6A). These higher serum TNF levels correlated with higher TNF levels in the liver but not in the lung or the heart. These results reveal a fundamental difference between the systemic inflammatory responses in endotoxemia and hemorrhage. The pattern of TNF expression during resuscitation in splenectomized animals is similar to that of non-splenectomized animals. More significant, GTS significantly inhibited systemic TNF levels in splenectomized animals (FIG. 6B). However, the anti-inflammatory potential of GTS was lower than in non-splenectomized animals particularly in the liver. However, unlike vagus nerve stimulation or treatment with nicotine, GTS significantly inhibited serum TNF levels as well as TNF levels in the lung and the heart of splenectomized animals. These results indicate that GTS might provide a significant advantage as compared with non-specific cholinergic agonists to control systemic inflammation particularly in those patients with injured or compromised spleen.

Alpha7nAChR-Agonist Inhibited Systemic HMGB1 Response and PARP Activation During Hemorrhage

HMGB1 is a characteristic marker of cellular damage that can act as a cardiodepressant factor causing acute lung injury and abrupt cardiac standstill. (FIG. 7, Alpha7nAChR-agonist inhibits systemic HMGB1 response. (A) Blood from control or hemorrhagic animals with or without resuscitation were analyzed at 2 hrs after hemorrhage to determine serum HMGB1 levels. (B) GTS inhibits PARP activity in the liver, a major source of HMGB1 during hemorrhage. * represents P<0.05 versus control (n=4/group; one-way ANOVA with Bonferroni's corrections)). Hemorrhage induced a systemic HMGB1 response that was not prevented by resuscitation with Hextend (FIG. 7A). However, one of the most significant effects of GTS was inhibiting systemic HMGB1 response during resuscitation. These results were particularly significant as no serum HMGB1 was found in those animals resuscitated with Hextend containing GTS. Because recent studies associated HMGB1 with liver damage during reperfusion and PARP appears to regulate HMGB1 secretion and contributes to hemorrhagic shock, we analyzed the effects of GTS on hepatic PARP activity. Hemorrhage activates hepatic PARP, and resuscitation with Hextend did not prevent it. However, resuscitation with Hextend supplemented with GTS inhibited PARP activation (FIG. 7B). Similar results were observed in other organs such as the spleen (data not shown). It is noteworthy that both serum HMGB1 levels and hepatic PARP activity correlated and the activity levels in animals resuscitated with GTS were similar to that in control animals.

### REFERENCES

1. Ulloa L. The vagus nerve and the nicotinic anti-inflammatory pathway. Nat Rev Drug Discov. 2005; 4: 673-84.
2. Ulloa L, Tracey K J. The “cytokine profile”: a code for sepsis. Trends Mol Med. 2005; 11: 56-63.
3. Ulloa L, Doody J, Massague J. Inhibition of transforming growth factor-beta/SMAD signalling by the interferon-gamma/STAT pathway. Nature. 1999; 397: 710-3.
4. Tracey K J, Cerami A. Tumor necrosis factor: a pleiotropic cytokine and therapeutic target. Annu Rev Med. 1994; 45: 491-503.
5. Ulloa L, Messmer D. High-mobility group box 1 (HMGB1) protein: friend and foe. Cytokine Growth Factor Rev. 2006; 17: 189-201.
6. Parrish W, Ulloa L. High-mobility group box-1 isoforms as potential therapeutic targets in sepsis. Methods Mol Biol. 2007; 361: 145-62.
7. Mantell L L, Parrish W R, Ulloa L. HMGB1 as a therapeutic target for infectious and inflammatory disorders. Shock. 2006; 25: 4-11.
8. Li W, Sama A E, Wang H. Role of HMGB1 in cardiovascular diseases. Curr Opin Pharmacol. 2006; 6: 130-5.
9. Sappington P L, Yang R, Yang H, Tracey K J, Delude R L, Fink M P. HMGB1 B box increases the permeability of Caco-2 enterocytic monolayers and impairs intestinal barrier function in mice. Gastroenterology. 2002; 123: 790-802.
10. Abraham E, Arcaroli J, Carmody A, Wang H, Tracey K J. HMG-1 as a mediator of acute lung inflammation. J. Immunol. 2000; 165: 2950-4.
11. Ulloa L, Wang P. The neuronal strategy for inflammation. Novartis Found Symp. 2007; 280: 223-33; discussion 233-7.
12. Wang H, Liao H, Ochani M, Justiniani M, Lin X, Yang L, Metz C, Miller E J, Tracey K J, Ulloa L. Cholinergic agonists inhibit HMGB1 release and improve survival in experimental sepsis. Nat Med. 2004; 10: 1216-21.
13. de Jonge W J, Ulloa L. The alpha7 nicotinic acetylcholine receptor as a pharmacological target for inflammation. Br J. Pharmacol. 2007; 151: 915-29.
14. Wang H, Yu M, Ochani M, Amella C A, Tanovic M, Susarla S, Li J R, Yang H, Ulloa L, Al-Abed Y, Czura C J, Tracey K J. Nicotinic acetylcholine receptor alpha7 subunit is an essential regulator of inflammation. Nature. 2003; 421: 384-8.
15. Huston J M, Ochani M, Rosas-Ballina M, Ochani K, Pavlov V A, Gallowitsch-Puerta M, Czura C J, Foxwell B, Tracey K J, Ulloa L. Splenectomy inactivates the cholinergic antiinflammatory pathway during lethal endotoxemia and polymicrobial sepsis. J Exp Med. 2006; 203: 1623-8.
16. Matthay M A, Ware L B. Can nicotine treat sepsis? Nat Med. 2004; 10: 1161-2.
17. Pullan R D, Rhodes J, Ganesh S, Mani V, Morris J S, Williams G T, Newcombe R G, Russell M A, Feyerabend C, Thomas G A, et al. Transdermal nicotine for active ulcerative colitis. N Engl J. Med. 1994; 330: 811-5.
18. Nanri M, Kasahara N, Yamamoto J, Miyake H, Watanabe H. A comparative study on the effects of nicotine and GTS-21, a new nicotinic agonist, on the locomotor activity and brain monoamine level. Jpn J Pharmacol. 1998; 78: 385-9.
19. Meyer E M, Kuryatov A, Gerzanich V, Lindstrom J, Papke R L. Analysis of 3-(4-hydroxy, 2-Methoxybenzylidene)anaba-seine selectivity and activity at human and rat alpha-7 nicotinic receptors. J Pharmacol Exp Ther. 1998; 287: 918-25.
20. Conejero-Goldberg C, Davies P, Ulloa L. Alpha7 nicotinic acetylcholine receptor: a link between inflammation and neurodegeneration. Neurosci Biobehav Rev. 2008; 32: 693-706.
21. Nielsen V G, Tan S, Brix A E, Baird M S, Parks D A. Hextend (hetastarch solution) decreases multiple organ injury and xanthine oxidase release after hepatoenteric ischemia-reperfusion in rabbits. Crit Care Med. 1997; 25: 1565-74.
22. Kellum J A. Fluid resuscitation and hyperchloremic acidosis in experimental sepsis: improved short-term survival and acid-base balance with Hextend compared with saline. Crit Care Med. 2002; 30: 300-5.
23. Gan T J, Bennett-Guerrero E, Phillips-Bute B, Wakeling H, Moskowitz D M, Olufolabi Y, Konstadt S N, Bradford C, Glass P S, Machin S J, Mythen M G. Hextend, a physiologically balanced plasma expander for large volume use in major surgery: a randomized phase III clinical trial. Hextend Study Group. Anesth Analg. 1999; 88: 992-8.
24. Handrigan M T, Bentley T B, Oliver J D, Tabaku L S, Burge J R, Atkins J L. Choice of fluid influences outcome in prolonged hypotensive resuscitation after hemorrhage in awake rats. Shock. 2005; 23: 337-43.
25. Messmer D, Yang H, Telusma G, Knoll F, Li J, Messmer B, Tracey K J, Chiorazzi N. High mobility group box protein 1: an endogenous signal for dendritic cell maturation and Th1 polarization. J. Immunol. 2004; 173: 307-13.
26. Tsung A, Sahai R, Tanaka H, Nakao A, Fink M P, Lotze M T, Yang H, Li J, Tracey K J, Geller D A, Billiar T R. The nuclear factor HMGB1 mediates hepatic injury after murine liver ischemia-reperfusion. J Exp Med. 2005; 201: 1135-43.
27. Ditsworth D, Zong W X, Thompson C B. Activation of poly(ADP)-ribose polymerase (PARP-1) induces release of the pro-inflammatory mediator HMGB1 from the nucleus. J Biol Chem. 2007; 282: 17845-54.
28. Szabo C. Potential role of the peroxynitrate-poly(ADP-ribose) synthetase pathway in a rat model of severe hemorrhagic shock. Shock. 1998; 9: 341-4.
29. Virag L, Szabo C. The therapeutic potential of poly(ADP-ribose) polymerase inhibitors. Pharmacol Rev. 2002; 54: 375-429.
30. Jagtap P, Szabo C. Poly(ADP-ribose) polymerase and the therapeutic effects of its inhibitors. Nat Rev Drug Discov. 2005; 4: 421-40.
31. Tapper A R, McKinney S L, Nashmi R, Schwarz J, Deshpande P, Labarca C, Whiteaker P, Marks M J, Collins A C, Lester H A. Nicotine activation of alpha4* receptors: sufficient for reward, tolerance, and sensitization. Science. 2004; 306: 1029-32.
32. Villegier A S, Salomon L, Granon S, Changeux J P, Belluzzi J D, Leslie F M, Tassin J P. Monoamine oxidase inhibitors allow locomotor and rewarding responses to nicotine. Neuropsychopharmacology. 2005.
33. West K A, Brognard J, Clark A S, Linnoila I R, Yang X, Swain S M, Harris C, Belinsky S, Dennis P A. Rapid Akt activation by nicotine and a tobacco carcinogen modulates the phenotype of normal human airway epithelial cells. J Clin Invest. 2003; 111: 81-90.
34. Vincler M A, Eisenach J C. Knock down of the alpha 5 nicotinic acetylcholine receptor in spinal nerve-ligated rats alleviates mechanical allodynia. Pharmacol Biochem Behav. 2005; 80: 135-43.
35. van Westerloo D J, Giebelen I A, Florquin S, Bruno M J, Larosa G J, Ulloa L, Tracey K J, van der Poll T. The vagus nerve and nicotinic receptors modulate experimental pancreatitis severity in mice. Gastroenterology. 2006; 130: 1822-30.
36. Yeboah M M, Xue X, Duan B, Ochani M, Tracey K J, Sushi M, Metz C N. Cholinergic agonists attenuate renal ischemic-reperfusion injury in rats. Kidney Int. 2008.
37. Wang H, Bloom O, Zhang M, Vishnubhakat J M, Ombrellino M, Che J, Frazier A, Yang H, Ivanova S, Borovikova L, Manogue K R, Faist E, Abraham E, Andersson J, Andersson U, Molina P E, Abumrad N N, Sama A, Tracey K J. HMG-1 as a late mediator of endotoxin lethality in mice. Science. 1999; 285: 248-51.
38. Lotze M T, Tracey K J. High-mobility group box 1 protein (HMGB1): nuclear weapon in the immune arsenal. Nat Rev Immunol. 2005; 5: 331-42.
39. Ombrellino M, Wang H, Ajemian M S, Talhouk A, Scher L A, Friedman S G, Tracey K J. Increased serum concentrations of high-mobility-group protein 1 in haemorrhagic shock. Lancet. 1999; 354 1446-7.
40. Ulloa L, Ochani M, Yang H, Tanovic M, Halperin D, Yang R, Czura C J, Fink M P, Tracey K J. Ethyl pyruvate prevents lethality in mice with established lethal sepsis and systemic inflammation. Proc Natl Acad Sci USA. 2002; 99: 12351-6.
41. Ulloa L, Fink M P, Tracey K J. Ethyl pyruvate protects against lethal systemic inflammation by preventing HMGB1 release. Aim. NY Acad. Sci. 2003; 987: 319-21.
42. Gardella S, Andrei C, Ferrera D, Lotti L V, Torrisi M R, Bianchi M E, Rubartelli A. The nuclear protein HMGB1 is secreted by monocytes via a non-classical, vesiclemediated secretory pathway. EMBO Rep. 2002; 3: 995-1001.
43. Scaffidi P, Misteli T, Bianchi M E. Release of chromatin protein HMGB1 by necrotic cells triggers inflammation. Nature. 2002; 418: 191-5.
44. Rubartelli A, Lotze M T. Inside, outside, upside down: damage-associated molecular-pattern molecules (DAMPs) and redox. Trends Immunol. 2007; 28: 429-36.
45. Matzinger P. The danger model: a renewed sense of self. Science. 2002; 296: 301-5.
46. Dumitriu I E, Baruah P, Manfredi A A, Bianchi M E, Rovere-Querini P. HMGB1: guiding immunity from within. Trends Immunol. 2005; 26: 381-7.
47. Akira S, Takeda K. Toll-like receptor signaling. Nat Rev Immunol. 2004; 4: 499-511.
48. Liaudet L, Soriano F G, Szabo E, Virag L, Mabley J G, Salzman A L, Szabo C. Protection against hemorrhagic shock in mice genetically deficient in poly(ADPribose) polymerase. Proc Natl Acad Sci USA. 2000; 97: 10203-8.
49. Virag L, Scott G S, Cuzzocrea S, Manner D, Salzman A L, Szabo C. Peroxynitriteinduced thymocyte apoptosis: the role of caspases and poly (ADP-ribose) synthetase (PARS) activation. Immunology. 1998; 94: 345-55.
50. Ha H C, Snyder S H. Poly(ADP-ribose) polymerase is a mediator of necrotic cell death by ATP depletion. Proc Natl Acad Sci USA. 1999; 96: 13978-82.

1. A method of controlling systemic inflammation during resuscitation and improving survival in a patient suffering a severe hemorrhage which comprises administering an α7nAChR-agonist to the patient.

2. The method of claim 1 wherein the α7nAChR-agonist is GTS.
